(19) United States
(12) Patent Application Publication
(10) Pub. No.: US 2009/0264786 A1
(43) Pub. Date: Oct. 22, 2009

(54) System and Method For Signal Denoising Using Independent Component Analysis and Fractal Dimension Estimation

(75) Inventor: Arnaud Jacquin, New York, NY (US)

Correspondence Address: FINNEGAN, HENDERSON, FARABOW, GARRETT & DUNNER;LLP, 901 NEW YORK AVENUE, NW, WASHINGTON, DC, 20001-4413, US

(73) Assignee: BrainScope Company, Inc.

(21) Appl. No.: 12/106,657

(22) Filed: Apr. 21, 2008

(51) Int. Cl. A61B 5/055; G06F 17/10

(52) U.S. Cl. 600/544; 708/323

(57) ABSTRACT

A system and method of signal denoising using Independent Component Analysis (ICA) and fractal dimension analysis of the signal components in the ICA domain is described. The signal components with fractal dimensions higher than a pre-determined threshold are automatically attenuated or canceled in order to alleviate the noise in the signal. The denoised signal is reconstructed using inverse ICA transform of the signal components.

### FIELD OF THE INVENTION

This invention relates to the field of signal denoising, and more particularly, to a method and apparatus for brain electrical signal acquisition, and automatic, real-time cancellation of artifacts from the acquired signals.

### BACKGROUND OF THE INVENTION

Denoising, the restoration of distorted or noisy signals, is an ongoing challenge of signal processing. One of the most rampant causes of signal noise is the additive white Gaussian noise which can be caused by poor data acquisition or by transmission of data in noisy communication channels. Early methods of signal denoising involved signal averaging to minimize noise, or linear filtering to smooth out the high-frequency regions generally associated with noise.

Newer and better approaches perform some thresholding in the wavelet domain of a signal, which attempts to remove whatever noise is present and retain whatever signal is present regardless of the frequency content of the signal. In this method, the data is at first decomposed using wavelet transform, all frequency sub-band coefficients that have a magnitude lower than a pre-determined threshold are set to zero, and an inverse wavelet transformation is then performed to reconstruct the data set. However, thresholding of all low magnitude coefficients can lead to omission of certain relevant details of the data set. Another inherent problem with this method is the choice of a suitable threshold value. Most signals show a non-uniform energy distribution, and hence, a noisy input signal may consist of parts where the magnitude of the signal are below the globally defined threshold and other parts where the noise magnitudes exceed the set threshold. Therefore, if the denoising methodology relies solely on a globally defined threshold, it can omit relevant parts of the signals on one hand, and leave some noise intact on the other.

More recently, this denoising method has been enhanced by performing soft-thresholding, wherein the wavelet coefficients are shrinked (non-linear soft thresholding) according to noise variation estimation. However, to achieve optimal results, the wavelet shrinkage denoising technique requires a priori knowledge of the noise and the signal to be retrieved to select a data-adaptive threshold, and therefore, is not practical for real-world experiments.

In recent years, various source separation algorithms have been developed that are optimized to correct or remove signal contaminates. These algorithms make minimal assumptions about the underlying process, thus approaching in some aspects, blind source separation (BSS) techniques. These techniques are based on the “unmixing” of the input signal into some number of underlying components using a signal separation algorithm, such as Independent Component Analysis, Principle Component Analysis, etc., followed by “remixing” only those components that would result in a “clean” signal by nullifying the weight of unwanted components.

The recognition and cancellation of components that generate artifacts is, however, a delicate, complicated and sometimes tedious task, and is often performed by a human expert. There is currently no known method of automatic identification and cancellation of signal components that are contaminated by noise.

### SUMMARY OF THE INVENTION

It is a primary object of the invention to present a technique for automatic detection and rejection of signal artifacts without requiring individual manual adjustment. In an exemplary embodiment of the invention, this is achieved by using a fractal dimension-based analysis of the signal components. The signal is at first decomposed into a plurality of signal components using a signal transform process. The fractal dimensions of the signal components are then computed in the transform domain. Based on the fractal dimension estimates, noise components are identified and modified. A denoised signal is then reconstructed using an inverse transform.

In accordance with an exemplary embodiment, there is provided a method of signal denoising wherein a given signal is deconstructed into its sub-components using Independent Component Analysis (ICA), which is a computational and statistical technique for separating a multivariate signal into its additive subcomponents, supposing that the source signals are non-Gaussian and mutually independent. The fractal dimensions of the signal components are then calculated, and the components that have a fractal dimension higher than a threshold value are automatically canceled, attenuated to a non-zero value, or otherwise modified. A denoised signal is then reconstructed with the intact and modified components using an inverse transform.

Essentially, signal components having high fractal dimensions are generally associated with noise. In an exemplary embodiment, by attenuating these components, the noise is in effect reduced. The components are then remixed using an inverse operation to generate a cleaner signal, which can then be subjected to downstream signal analysis and/or other information processing.

In accordance with an exemplary embodiment of the invention, there is provided a system of signal denoising comprising the steps of source separation using Independent Component Analysis (ICA), identification of noise components using fractal dimension analysis in the source/component space, processing the identified noise components, and reprojection of the components into the signal space using inverse ICA transform.

In accordance with a further exemplary embodiment of the present invention, there is provided a system for denoising brain electrical signals comprising the steps of source (component) separation using ICA, identification of noise components in the source/component domain using fractal dimension analysis, attenuation of the identified noise components, and reprojection of the components into the signal space using inverse ICA transform.

In accordance with a further illustrative embodiment of the present invention, there is provided an apparatus for practicing the invention, which can be embodied in the form of a computer program code containing instructions, which can either be stored in a computer readable storage medium such as floppy disks, CD-ROMs, hard drives etc., or can be transmitted over the internet, such that, when the computer program code is loaded into and executed by an electronic device such as a computer, a microprocessor or a microcontroller, the device and its peripheral modules become an apparatus for practicing the invention.

Additional objects and advantages of the invention will be set forth in part in the description which follows, and in part will be obvious from the description, or may be learned by practice of the invention. The objects and advantages of the invention will

It is to be understood that both the foregoing general description and the following detailed description are exemplary and explanatory only and are not restrictive of the invention, as claimed.

The accompanying drawings, which are incorporated in and constitute a part of this specification, illustrate embodiments of the invention and together with the description, serve to explain the principles of the various aspects of the invention.

### BRIEF DESCRIPTION OF THE DRAWINGS

FIG. 1 is a flowchart illustrating the method of signal denoising carried out by a device according to an exemplary embodiment of the present invention.

FIG. 2A is diagram illustrating noisy brain electrical activity, and the decomposition of the recorded signals into independent sources using ICA.

FIG. 2B is diagram illustrating the removal of Electromyographic (EMG) artifacts from recorded brain electrical activity without removing the underlying brain-generated signals.

FIG. 3 is a diagram illustrating an apparatus for recording and denoising brain electrical signals according to an exemplary embodiment consistent with the present invention.

### DESCRIPTION OF ILLUSTRATIVE EMBODIMENTS

Reference will now be made in detail to exemplary embodiments of the invention, examples of which are illustrated in the accompanying drawings. Wherever possible, the same reference numbers will be used throughout the drawings to refer to the same or like parts.

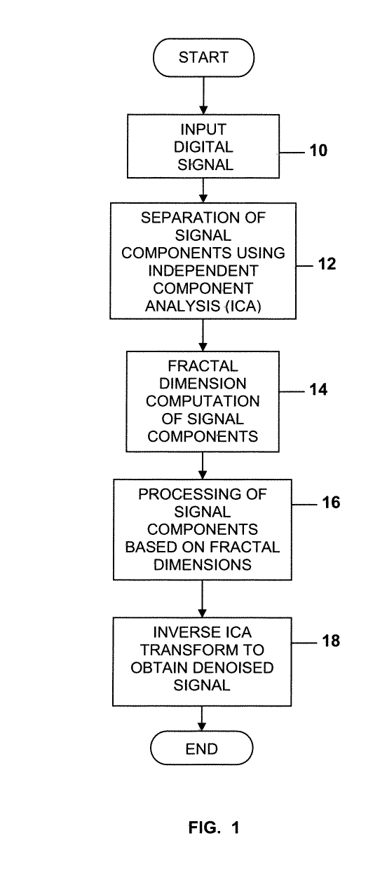

In accordance with embodiments consistent with the present invention, FIG. 1 shows a flowchart illustrating a method of signal denoising. This method may be implemented by an electronic device, such as a computer or a microprocessor, which has the instructions for performing the method loaded into its memory. A digital signal is entered into the signal processor (step 10). The signal can originate as an analog signal and can be converted to a digital signal by known means, or the signal may originate as a digital signal as would be understood by one of ordinary skill in the art. The signal is then separated into its sources or components using ICA (step 12). In an illustrative embodiment of the present invention, the FastICA algorithm invented by Aapo Hyvärinen is used (A. Hyvärinen, Neurocomputing 22, 1998, 49-67), which is incorporated herein by reference in its entirety. However, any other ICA algorithm, such as Infomax, JADE etc., may be applied for step 12. The basic premise of ICA is the assumption that the observed signals X=(X₁, . . . , X_{N}) recorded at N locations are the result of linear mixing of N source signals S=(S₁, . . . , S_{N}), such that X=MS, where M is a N×N mixing matrix estimated by the ICA algorithm. Thus, decomposing the observed signals X is akin to separating the source signals S. The source signals are given by the operation:

S=M⁻¹X,

where M⁻¹ is the N×N unmixing matrix given by the inverse of the mixing matrix.

Referring again to FIG. 1, the fractal dimensions of the components/sources are then computed (step 14) using the algorithm proposed by Higuchi (T. Higuchi, Physica D 31, 1988, 277-238), which is incorporated herein by reference in its entirety. However, any other algorithm for estimating fractal dimensions may also be used. The fractal dimension D of a signal is a measure of its “irregularity” or “complexity”. Unlike many estimates of the fractal dimension, the estimator proposed by Higuchi has the advantage of having low computational complexity, along with giving reliable estimates with as few as 100 data points. Higuchi's estimates of the fractal dimension of a one dimensional signal yields values close to 1 for smooth signals, and for random noise it generates a value close to 2, which is the theoretical maximum for a one dimensional signal.

The signal components with D higher than a preset threshold value are automatically attenuated or canceled (step 16). This process of signal de-noising is a non-linear operation as different components are affected differently by the attenuation or cancellation process. The de-noised signal is then reconstructed by computing the inverse transform (step 18), and can then be subjected to signal analysis and/or other information processing. The denoised signal X_{d} is obtained as:

X_{d}=MQS,

where Q is a non-linear operator that processes one component Sₖ (i.e. k^{th} component of S) at a time in the component/source domain. The component Sₖ is left intact if it has a fractal dimension lower than a predetermined threshold value. If its fractal dimension is higher than the threshold, it is assumed to correspond to noise artifacts, and gets canceled, de-emphasized, or otherwise modified.

This method of signal processing allows effective denoising using fewer data points, and thereby allows much faster acquisition of denoised data sets to be used for signal analysis. This is particular important for applications where immediate results are sought, as in the case of near real-time medical diagnostic tests in the emergency department or in an ambulatory setting.

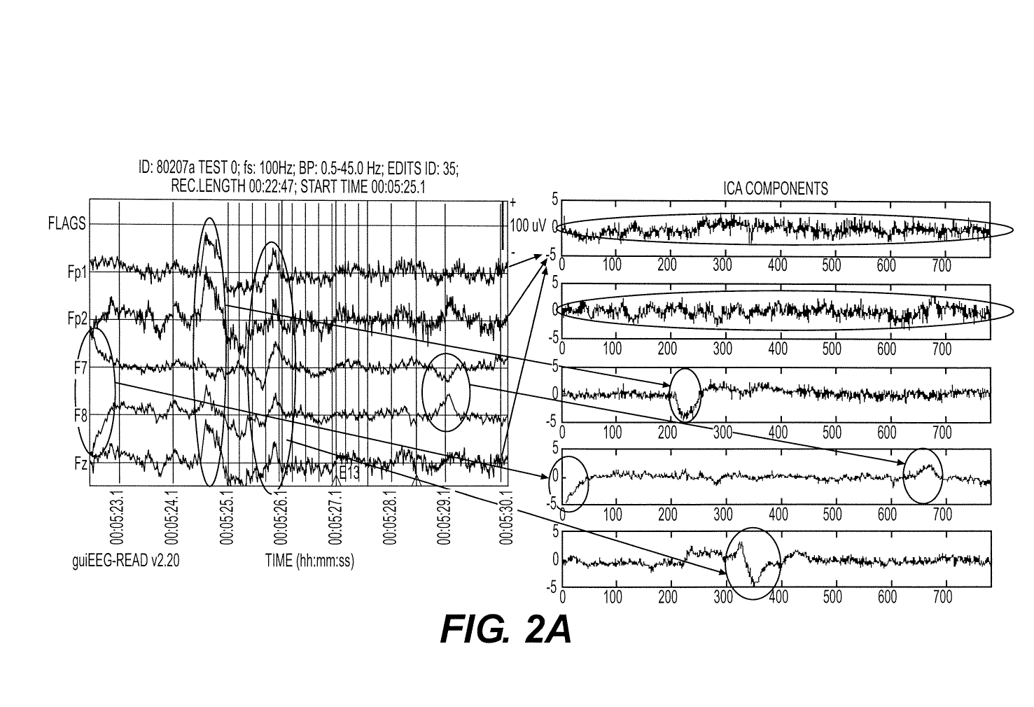

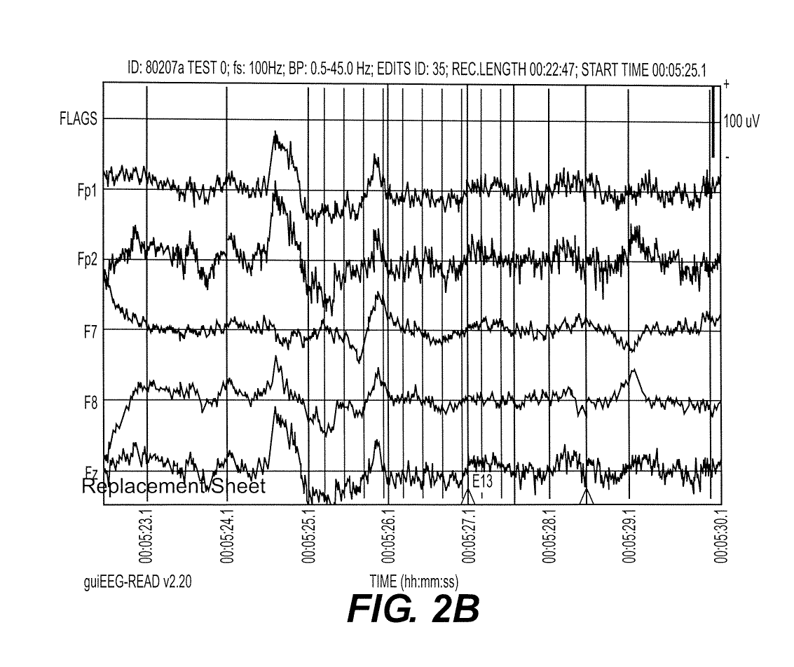

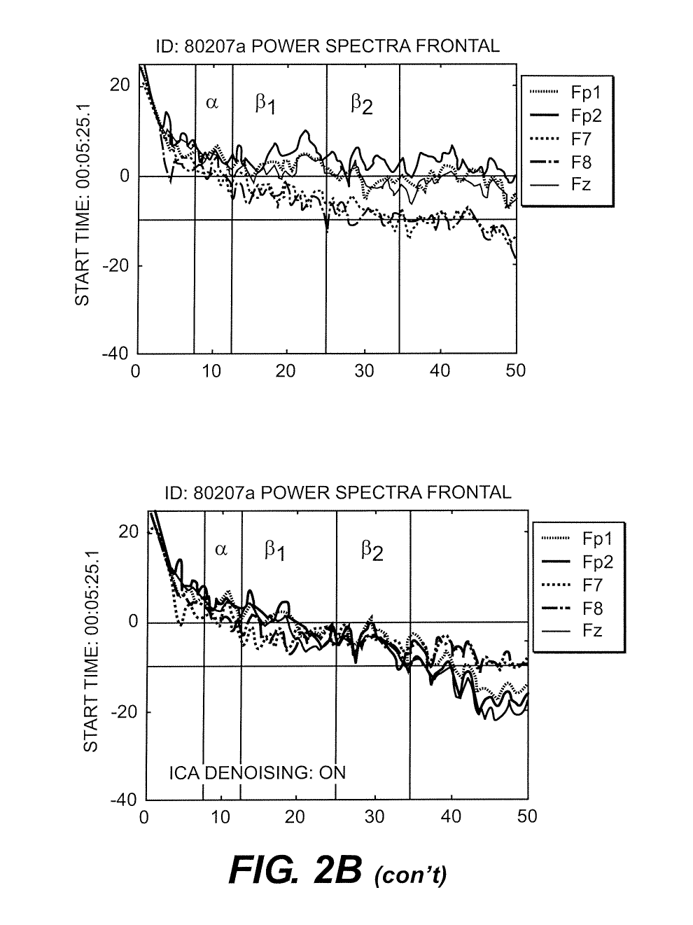

In an exemplary embodiment consistent with the present invention, the denoising technique described above is used for artifact subtraction in brain electrical activity. FIG. 2A shows the brain electrical signal recorded at 5 electrode locations, and the source/components separated by the ICA algorithm. The ICA is performed on three epochs of 2.56 seconds length (256 data points) to create a padded epoch of 768 data points total in order to avoid edge effects. Fractal dimension is then computed over segments of 1.28 seconds in the ICA component domain. The fractal dimension D may be divided into the following ranges:

0≦D≦1.8 1)

1.8≦D≦1.9 2)

D≧1.9 3)

The signal components with D higher than a preset threshold value are then automatically attenuated using a low-pass filter. For example, for the removal of Electromyographic (EMG) artifacts, generated due to subject tension/nervousness, a threshold value of 1.8 is selected, and the components with fractal dimension higher than 1.8 (cases 2 and 3, for example) are attenuated. The denoised signal is then reconstructed using an inverse transform of the intact and attenuated components. FIG. 2B shows the signal with EMG artifacts removed without affecting the brain-generated signals. As further shown in FIG. 2B, denoising by the fractal dimension analysis methodology described herein does not appreciably degrade the power spectral content of the brain electrical signals. The denoising process also speeds up the acquisition of clean data epochs for downstream signal analysis.

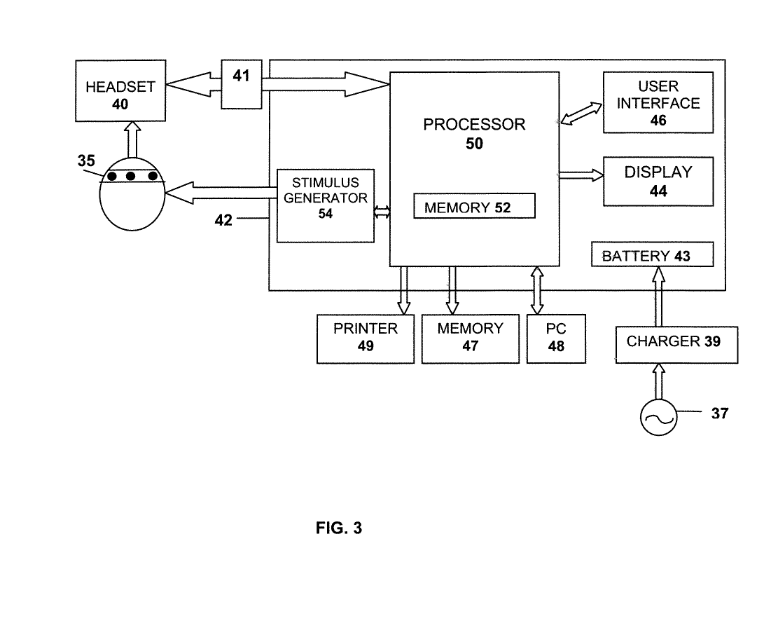

In accordance with embodiments consistent with the present invention, FIG. 3 shows an apparatus for acquiring and denoising brain electrical signals using BX™ technology. This apparatus consists of a headset 40 which may be coupled to a base unit 42, which can be handheld, as illustrated in FIG. 3. The headset 40 may include a plurality of electrodes 35 to be attached to a subject's head.

The base unit 42 may include a display 44, which can be a LCD screen, and can further have a user interface 46, which can be a touch screen user interface or a traditional key-board type interface. The interface 41 can act as a multi-channel input/output interface for the headset 40 and the handheld device 42, to facilitate bidirectional communication of signals to and from the processor 50, such that a command from the user entered through the user interface 46 can start the signal acquisition process of headset 40. Interface 41 may include a permanently attached or detachable cable or wire, or may include a wireless transceiver, capable of wirelessly transmitting and receiving signals from the headset, or from an external device storing captured signals. In an embodiment consistent with the present invention and in accordance with the Bx™ technology, the headset 40 can include analog amplification channels connected to the electrodes, and an analog-to-digital converter (ADC) to digitize the acquired brain electrical signals prior to receipt by the base unit 42.

In an exemplary embodiment consistent with the present invention, noise artifacts are removed from the acquired signal in the signal processor 50, which performs a de-noising method as described above and illustrated in FIG. 1, as per instructions loaded into memory 52. The memory 52 may further contain interactive instructions for using and operating the device to be displayed on the screen 44. The instructions may comprise an interactive feature-rich presentation including a multimedia recording providing audio/video instructions for operating the device, or alternatively simple text, displayed on the screen, illustrating step-by-step instructions for operating and using the device. The inclusion of interactive instructions with the device eliminates the need for a device that requires extensive training to use, allowing for deployment and use by persons other than medical professionals.

The denoised signal may be further processed in the processor 50 to extract signal features, and the output maybe displayed on the display 44, or may be saved in external memory or storage 47, or may be displayed on a PC 48 connected to the base unit 42. In one embodiment, the results can be transmitted wirelessly or via a cable to a printer 49 that prints the results. Base unit 42 also contains an internal rechargeable battery 43 that can be charged during or in between uses by battery charger 39 connected to an AC outlet 37. The battery can also be charged wirelessly through electromagnetic coupling by methods known in the prior art, in which case the base unit 42 would also contain an antenna for receiving the RF emission from an external source. In further accordance with BX™ technology, base unit 42 may also contain a wireless power amplifier coupled to an antenna to transmit the results wirelessly to PC 48 or an external memory 47 store the results.

In another embodiment consistent with the present invention, the processor 50 transmits the raw, unprocessed signal to the computer 48. The computer performs the de-noising method illustrated in FIG. 1, and optionally further analyzes the signal and output the results.

In one embodiment, the headset 40 and the base unit 42 along with the charger 39 may come as a kit for field use or point-of-care applications. In yet another embodiment consistent with the present invention, both the headset 40 and the base unit 42 may be configured to reside on a common platform, such as a headband, to be attached to the subject's head. In further accordance with Bx™ technology, the processor of the base unit, and the analog amplification channels and ADC of the headset may be configured to reside on a single integrated physical circuit.

In yet another embodiment consistent with the present invention, the base unit 42 includes a stimulus generator 54 for applying stimuli (e.g. electrical, tactile, acoustic stimuli etc.) to the subject to elicit evoked potentials. The processor 50 then denoises and further analyzes both the spontaneous brain electrical signals as well as evoked potentials generated in response to the applied stimuli.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A method for signal denoising, comprising the steps of:
i. decomposing the signal into a plurality of independent signal components using a signal transform;
ii. computing fractal dimensions of the components in the transform domain;
iii. identifying noise components based on their fractal dimensions;
iv. modifying the identified noise components;
V. reconstructing a denoised signal using inverse transform.

2. The method of claim 1, wherein the signal is decomposed into a plurality of independent signal components using Independent Component Analysis (ICA).

3. The method of claim 1, wherein the step of identifying noise components is performed automatically.

4. The method of claim 1, wherein the step of modifying comprises attenuation of signal components having a fractal dimension higher than a threshold value.

5. The method of claim 4, wherein the threshold value is predetermined.

6. The method of claim 4, wherein the attenuation is a non-linear process.

7. The method of claim 1, further comprising the step of automatically forwarding the denoised signal for further signal analysis.

8. A system for denoising a signal, the system comprising a processor configured for:
i. transforming the signal into a plurality of independent signal components;
ii. measuring the fractal dimensions of the components;
iii. processing the components with fractal dimensions higher than a predetermined value; and
iv. reconstructing a denoised signal using inverse transform.

9. The system of claim 8, wherein the processor is configured to separate the signal into a plurality of independent signal components using Independent Component Analysis (ICA).

10. The system of claim 9, wherein the processor is configured to reconstruct the denoised signal using inverse ICA transform.

11. The system of claim 8, wherein the processor is configured to cancel signal components with fractal dimensions higher that a predetermined threshold.

12. The system of claim 8, wherein the processor is configured to attenuate signal components with fractal dimensions higher that a predetermined threshold.

13. The system of claim 11, wherein the processor is configured to reconstruct a denoised signal using inverse transform of remaining signal components.

14. The system of claim 12, wherein the processor is configured to reconstruct a denoised signal using inverse transform of intact and the attenuated signal components.

15. A system for denoising brain electrical signals, the system comprising a processor configured for:
i. separating the signals into a plurality of independent signal sources/components using Independent Component Analysis;
ii. measuring the fractal dimensions of the components;
iii. automatically attenuating the components with fractal dimensions higher than a predetermined value; and
iv. reconstructing a denoised signal using inverse ICA transform of the attenuated and intact components.

16. An apparatus for acquiring and denoising brain electrical signals of a subject, comprising:
a headset comprising at least one electrode;
a base unit; wherein
said base unit further comprises a processor configured to utilize one or more operating instructions to perform denoising of the received signal using Independent Component Analysis and fractal dimension analysis.

17. The apparatus of claim 16, wherein the processor is configured to further analyze the denoised signal and output a result.

18. The apparatus of claim 17, further comprising a display wherein the result of one or more operations performed by the processor is displayed.

19. The apparatus of claim 18, wherein the display is operatively connected to the processor; and
wherein the display can be integrated into the base unit, or can be external to the base unit.

20. The apparatus of claim 16, wherein the headset communicates wirelessly with the base unit.

21. The apparatus of claim 16, wherein the headset comprises at least one analog amplification channel.

22. The apparatus of claim 21, wherein the headset further comprises an analog-to-digital converter.

23. The apparatus of claim 16, wherein the base unit communicates wirelessly with an external display.

24. The apparatus of claim 16, wherein the base unit comprises a stimulus generator to apply stimuli to the subject; and
wherein the processor is configured to denoise spontaneous brain electrical signals and evoked potentials generated in response to the applied stimuli.

25. The apparatus of claim 22, wherein the headset and the base unit are configured to reside on a single platform to be connected to the subject; and
wherein the processor, the at least one analog amplification channel, and the analog-to-digital converter are configured to reside on a single integrated physical circuit.
